(19) United States
(12) Patent Application Publication
(10) Pub. No.: US 2010/0184012 A1
(43) Pub. Date: Jul. 22, 2010

(54) METHODS AND DEVICES FOR IMAGING AND MANIPULATING BIOLOGICAL SAMPLES

(76) Inventor: Mark A. Voelker, Emeryville, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/690,597

(22) Filed: Jan. 20, 2010

Related U.S. Application Data
(63) Continuation-in-part of application No. 11/601,434, filed on Nov. 17, 2006.
(60) Provisional application No. 60/789,541, filed on Apr. 4, 2006.

(51) Int. Cl. A01N 1/02; C12M 1/34; C12Q 1/02

(52) U.S. Cl. 435/2; 435/288.7; 435/29

(57) ABSTRACT

Methods of imaging biological samples are provided. Aspects of embodiments of the methods include freezing, thawing and imaging a biological sample, one or more times, in a manner sufficient to image the biological sample while maintaining the viability and/or structural integrity of the sample. Also provided are devices and systems for use in practicing the methods.

### CROSS-REFERENCE TO RELATED APPLICATIONS

This application is a continuation-in-part application of U.S. application Ser. No. 11/601,434 filed Nov. 17, 2006, which applications claims the priority benefit of U.S. provisional application No. 60/789,541, filed Apr. 4, 2006, which applications are incorporated herein by reference in their entirety.

### BACKGROUND

The development of microscopy has allowed scientists to image cells and tissues with increasing levels of detail and with increasing spatial and spectral resolution. Improvements in the detail that is visible in microscope images of cells and tissues have helped scientists understand how living organisms function and sometimes malfunction. This has increased the understanding of the structure and composition of various biological cells and tissues and has advanced the development of new protocols for the investigation, screening and diagnosis of disease.

Two techniques available for acquiring high-resolution images of biological samples include: optical microscopy and electron microscopy. Optical microscopy uses photon bombardment to magnify a sample. Optical microscopy allows scientists to image living cells and tissues with a spatial resolution traditionally defined by the Rayleigh criterion. In practical terms, the Rayleigh criterion means spatial resolution of at least about 200 nm for the best oil immersion objectives, but more typically up to about 500 nm for microscopes that do not reach Rayleigh criterion performance. Optical microscopes are easy to use, relatively inexpensive and can image living samples without killing them. However, the Rayleigh criterion spatial resolution attainable with optical microscopes is too large to directly image most of the molecular-scale components of living cells.

The electron microscope uses electron bombardment to magnify a sample. Using electron microscopy, biological cells can be imaged at very high spatial resolution (10 nm or better) and magnified over 2 million times. This allows for the direct imaging of cells and their components in minute detail. Intracellular structures, such as membranes, chromosomes, vesicles, microtubules, and even large protein molecules, may be imaged with the electron microscope. However, sample preparation methods, and the energetic nature of electron bombardment itself, usually causes loss of viability when electron microscopy is used to image biological samples. The tradeoff for being able to achieve such high resolution imaging of biological cells using the electron microscope is that the cells so imaged are killed in the process of acquiring the images.

There is continued interest in developing methods and devices for imaging the components of cells, tissues and organs with increasing levels of detail and with increasing spatial and spectral resolution.

### SUMMARY

Methods of imaging biological samples are provided. Aspects of embodiments of the methods include freezing, thawing and imaging a biological sample, one or more times, in a manner sufficient to image the biological sample, while maintaining the viability and/or structural integrity of the sample. Also provided are devices and systems for use in practicing the methods.

Methods of manipulating and imaging a manipulated biological sample are also provided. One embodiment of the methods includes repeatedly freezing, thawing and imaging a biological sample, in a manner sufficient to maintain the viability of the sample, wherein the sample may be manipulated once frozen or thawed. The biological sample may be imaged before, during or after the sample is manipulated, frozen and/or thawed.

In certain embodiments, the methods include thawing and/or manipulating a biological sample and imaging the thawed and/or manipulated sample. In certain embodiments, the methods include freezing and/or manipulating a frozen biological sample and imaging the frozen sample, wherein the sample has previously been thawed and/or manipulated and/or imaged. In certain embodiments, the methods include refreezing or thawing and imaging and/or manipulating a previously thawed or frozen viable biological sample wherein the sample has not previously been chemically fixed, stained, embedded, or otherwise treated in a manner that destroys the viability of the biological sample.

The methods of the invention are useful for repeatedly freezing, thawing, imaging and/or manipulating a viable biological sample. Once thawed or frozen the biological sample may be imaged and observed, for instance, via optical microscopy, and/or manipulated by contacting it with physical probes or radiation or chemical reagents or molecular nanodevices. In this manner, a biological sample may be repeatedly frozen, imaged and/or manipulated, thawed, and imaged and/or manipulated over a prolonged period of time while maintaining the viability and/or the structural integrity of the biological sample. For instance, while in the frozen state, the sample may be contacted for arbitrarily long periods of time with photons, electrons, physical, chemical, molecular or other probes, allowing intricate, detailed and precise observation, imaging and/or manipulation of the sample.

Also provided is an apparatus for performing the methods of the invention. An apparatus of the invention is configured for freezing and thawing a sample, for instance, a biological sample, under pressure. In certain embodiments, the apparatus is configured for being operated in conjunction with an apparatus for visualizing a frozen or thawed sample. In certain embodiments, the apparatus includes a chamber that includes an interior configured for holding a sample, a pressure modulator for modulating the pressure within the interior of the chamber and a temperature modulator for modulating the temperature of the interior of the chamber from a temperature that is below the freezing point of water to a temperature that is above the freezing point of water and vice-versa.

In certain embodiments, one or more of the components of the chamber are configured in such a manner so as to allow the transmission of photons, electrons and the like, from the outside of the chamber to the interior of the chamber, as well as to allow transmission of photons, electrons and the like, from the interior of the chamber to the outside of the chamber, to facilitate the observation of a sample within the chamber. In certain embodiments, the apparatus includes, in addition to the chamber, an imaging element, for instance, one or more devices configured for and positioned to contact the sample with photons, electrons, or the like, while the sample is inside the chamber and thereby image the sample. In certain other embodiments, the apparatus includes an imaging element that is configured for and positioned to contact the sample with photons, electrons, or the like, while the sample is outside the chamber. In certain embodiments, the apparatus includes one or more elements configured for and positioned to contact the sample with physical, chemical, molecular or other probes while the sample is inside or outside of the chamber and thereby manipulate the sample. In certain embodiments, the apparatus includes devices configured to add or subtract material from the sample while the sample is frozen or unfrozen sample and inside or outside the chamber.

### BRIEF DESCRIPTION OF THE FIGURES

FIG. 1 illustrates one embodiment of an apparatus of the invention.

FIG. 2 illustrates the embodiment of an apparatus of FIG. 1 of the invention with photon source, objective and upper and lower cones of light indicated.

FIG. 3 is an expanded cutaway view of the sample cell in FIG. 2.

FIG. 4 is an oblique view of the delivery manifold component in FIG. 1.

### DETAILED DESCRIPTION

Methods of imaging biological samples are provided. Aspects of embodiments of the methods include freezing, thawing and observing and/or imaging a biological sample, one or more times, in a manner sufficient to observe and/or image the biological sample while maintaining the viability and/or structural integrity of the sample. Also provided are devices and systems for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the stated ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference in their entirety as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

As summarized above, embodiments of the methods of the invention are directed to preparing and/or observing and/or imaging and/or manipulating a sample. An aspect of embodiments of the methods includes thawing and imaging a sample, for instance, a biological sample, in a manner sufficient to maintain the structural integrity of the biological sample. In certain embodiments, the biological sample is a viable biological sample and the methods of the invention include thawing and imaging the sample in a manner sufficient to maintain the viability of the biological sample. In certain embodiments, once thawed and/or imaged, the biological sample may be refrozen and/or imaged once frozen. For instance, the biological sample may be refrozen within a high pressure chamber and then manipulated and/or imaged in a manner sufficient to maintain the structural integrity and/or viability of the sample.

In certain embodiments, the methods of the invention involve the thawing of a frozen sample under pressure, for instance, high pressure, and the imaging of the sample once thawed. In certain embodiments, the methods of the invention also include the freezing or refreezing of a sample under pressure, for instance, high pressure, and imaging the sample once frozen. In one embodiment, the sample is imaged while under pressure, in a high pressure apparatus, such as the apparatus set forth in the Applicants' co-pending application Ser. No. 11/601,434 entitled Methods and Devices for Thawing a Frozen Sample, attorney docket number BIOT-009US1, which is herein incorporated by reference in its entirety. In other embodiments, the sample is imaged outside of a high pressure apparatus once the sample has been frozen and/or thawed within a high pressure apparatus.

Any sample can be frozen, observed and/or imaged and/or manipulated, thawed, observed and/or imaged and/or manipulated one or more times in accordance with the methods of the invention. For instance, the methods are suitable for use with an environmental sample or a biological sample, for instance, an organ, tissue, or cell sample. In certain embodiments, a sample includes multiple cells. In certain embodiments, a sample includes a multicellular organism. In certain embodiments, the sample is a viable biological sample. In certain embodiments, the sample may be one or more cells (e.g., a cell, a gamete cell, stem cell, or the like) that has been associated with a substrate, for instance, a glass, silicon or electronic chip. Further, the one or more cells may be arranged in a circuit configuration and may interact with other circuitry components to form a circuit that is capable of transferring a current or other signal from one point on the chip to another. In certain embodiments, the methods of the invention are characterized in that they are performed in such a manner and under conditions that preserve or maintain the viability of a biological sample. By “viability” of a biological sample is meant that the biological sample and/or one or more of its components maintains its ability to function, divide, differentiate, grow or otherwise live.

The sample may be any sample the analysis and/or modification of which is desired. The sample may be obtained from any suitable source in any manner sufficient to preserve the integrity of the sample, as is well known in the art. Where the sample is a biological sample it may be obtained from a suitable organ and/or tissue of interest. For instance, the sample may be a blood sample collected from a subject's veins via venipuncture, the sample may be an epidermal sample collected via skin grafting, the sample may be a tissue sample collected from some other organ (e.g., a liver, kidney, lungs, heart, brain or various other organs), the sample may be one or more ova, the sample may be one or more spermatozoa, the sample may be one or more embryos, the sample may be one or more embryonic or adult stem cells.

Another aspect of the invention is an apparatus for both freezing (e.g., cooling) and thawing (e.g., heating) a sample under pressure, for instance, high pressure, in a manner sufficient to reduce or prevent the formation of ice crystals within the sample caused by a thawing or freezing process that is not performed under high pressure. Hence, in certain embodiments, an apparatus of the invention is characterized in that it is configured for both freezing and thawing a biological sample contained inside a high pressure chamber of the apparatus in a manner sufficient to maintain the structural integrity and/or viability of the biological sample, and for observing and/or imaging and/or manipulating the sample, or for being associated with an apparatus for observing and/or imaging and/or manipulating the sample.

Where the sample is a biological sample, for instance, a cell or tissue sample or microorganism, the thawing, freezing, observing and/or imaging occur without substantially disrupting the structural integrity of the biological sample. Additionally, where the sample is a viable biological sample, the thawing, freezing, observing and/or imaging occur in a manner sufficient to maintain the viability of the sample.

The apparatus may be configured for observing and/or imaging the sample before, after or during the freezing and/or thawing process while the sample is inside or outside of a chamber and/or inside or outside of the apparatus. For instance, in certain embodiments, an apparatus of the invention allows observing and/or imaging and/or manipulating the sample while the sample is inside a sample containing element or high pressure chamber of the apparatus, e.g., the sample may be contained within a sample element that forms a high pressure chamber of the apparatus which is removed from the apparatus for observing and/or imaging and/or manipulation of the sample. In certain other embodiments, an apparatus of the invention may allow the sample to be removed temporarily from the sample containing element of the high pressure chamber of the apparatus for purposes of observing and/or imaging and/or manipulating the sample.

A suitable apparatus for use in the methods of the invention is set forth in the Applicants' co-pending application Attorney Docket Number [BIOT-009US-1]. In summary, a suitable apparatus for use in practicing the methods of the invention includes a chamber, a pressure modulator, a temperature modulator and an element for observation and/or imaging and/or manipulation.

The chamber includes an interior that is configured for holding a sample. For instance, where the pressure modulator includes two opposing surfaces (e.g. anvils) the chamber may be a cavity created between the two surfaces (e.g., anvils). Further, the chamber may be formed from the interior of a sample holding element that is adapted for holding a sample, for instance, a biological sample, and configured for being associated between the two surfaces (e.g., anvils) of the pressure modulator. For instance, the sample holding element may be a gasket, foil, membrane, or the like. The sample holding element may be fabricated out of any material (e.g., metal) so long as it is capable of associating with the opposing surfaces of the pressure modulator in a manner sufficient to withstand a high pressure generated by the pressure modulator. In certain embodiments, the sample holding element is a hard metal foil associated between two opposing surfaces and adapted for both holding a sample and supporting a contact point of the two surfaces. In certain embodiments, the chamber may contain a hydrostatic fluid.

The pressure modulator may be of any configuration so long as it is adapted for generating a pressure difference between the interior and the exterior of the chamber. In certain embodiments, the pressure modulator may include two opposing surfaces and a force generating mechanism (e.g., a compression mechanism). For instance, the pressure modulator may include two opposing surfaces (e.g., anvils) that are configured to form a chamber and/or associate with a sample holding element in a manner so as to form a chamber and are additionally operatively connected to a force generating mechanism in a manner sufficient to allow the two opposing surfaces to be compressed one toward the other which thereby generates a high pressure within the chamber.

The force generating mechanism which is operatively connected to the two surfaces may include one or more lever arms, screws, hydraulic systems or the like that are configured for being tightened or pressurized and thereby compressing the two opposing surfaces toward one another. The operative connection may be such that it generates a substantially uniaxial force that is applied to the base of the opposing surfaces thereby compressing the surfaces together and consequently generating a high pressure within the chamber.

In certain embodiments, the two opposing surfaces of the pressure modulator may be anvils. By “anvil” is meant a hard, fixed surface that is operatively connected with a force generating mechanism and configured for being compressed against a second hard, fixed surface and thereby generating a high pressure at the region of contact between the two surfaces. The anvils may be fabricated of any material capable of being compressed and withstanding the generation of a high pressure due to said compression without fracturing. For instance, the anvils may be diamonds, sapphires, or other precious or non-precious gem quality stones. Accordingly, a suitable device of the invention may be configured as a diamond anvil cell.

The temperature modulator may be of any configuration so long as it is adapted for modulating the temperature of the interior and/or exterior of the chamber. By “modulating the temperature of the interior and/or exterior of the chamber” is meant that the temperature modulator is capable of changing the temperature of the interior or exterior of the chamber from a first temperature to a second temperature. Accordingly, the temperature modulator controls the temperature of the interior of the chamber and is configured for changing the temperature within the chamber along a broad range of temperatures. Generally, the temperature modulator is configured for modulating the temperature of the interior in a range that includes a temperature that is below the freezing point of water to a temperature that is above the freezing point of water.

In certain embodiments, the temperature modulator includes a heating element. The heating element may be any means capable of generating and causing the transference of a high temperature (i.e., heat) to the interior of the chamber. For instance, a heating element may include a fluid, such as a gas or liquid that contacts the pressure modulator and/or chamber and thereby warms it. In certain embodiments, the heating element includes a helium gas or water that is heated and contacted with one or more of the opposing surfaces, e.g., anvils, of the pressure modulator.

In certain embodiments, the heating element is configured for contacting the pressure modulator with both a heated helium gas and a heated liquid, such as water. Accordingly, in these embodiments, the heating element is configured for heating the exterior components of the apparatus (e.g., the pressure modulator, anvils, sample holding element, gasket, etc.) which in turn transfers heat to the inside of the chamber and thereby warms the sample. In certain embodiments, the heating element may add heat directly to the inside of the chamber, for instance, by means of a resistive electrical element located inside the sample chamber. In certain embodiments, the heating element may add heat directly to the anvils, for instance by passing electrical current through anvils that are made of electrically conductive or semiconductive material, or by heating the anvils and/or the sample by means of magnetic inductive heating. In certain embodiments, the heating element may operate by irradiating the sample and/or the anvils with light or microwave energy or other electromagnetic energy which is absorbed by the material of the sample and/or the anvils. In certain embodiments, the heating element may operate by means of adiabatic magnetization of the anvils and/or the sample. Accordingly, in these embodiments, the heating element is configured for heating the interior or interior components of the apparatus (e.g., of the sample, anvils, sample holding element, gasket, etc.). In certain embodiments, the method of heating combines more than one of the methods described above (e.g. resistive heating and irradiation with electromagnetic energy).

In certain embodiments, the temperature modulator includes a cooling element. The cooling element may be any means capable of withdrawing heat from the interior of the chamber. For instance, a cooling element may include a fluid, such as a gas or liquid that contacts the pressure modulator and/or chamber. In certain embodiments, the cooling element includes a cryogenic liquid, for instance, liquid nitrogen that is contacted with one or more of the opposing surfaces, e.g., anvils, of the pressure modulator. Accordingly, in these embodiments the cooling element is configured for cooling the exterior components of the apparatus (e.g., the pressure modulator, sample holding element, etc.) that in turn cool the inside of the chamber and thereby freeze the sample, for instance, under high pressure. In certain embodiments, the cooling element is configured for cooling the interior of the interior components of the apparatus. For instance, the cooling element may remove heat from the sample or from the anvils by means of adiabatic demagnetization of the anvils, or of the sample. In certain embodiments, the cooling element may operate by means of laser or optical cooling, as in the manner of the Los Alamos Solid State Optical Refrigerator. In this embodiment, the anvils may be made of glass doped with Ytterbium, or other suitable compounds. In certain embodiments, the method of cooling combines more than one of the methods described above (e.g. adiabatic demagnetization and optical cooling).

In certain embodiments, the imaging element may be any element that is capable of imaging a sample while it is either inside or outside of a chamber of the apparatus. Accordingly, the imaging element may be an element intimately associated with and/or integrated with the pressure chamber of the invention or the imaging element may be a stand alone element stationed within proximity to the pressure chamber.

For instance, in certain embodiments, the apparatus includes, or is otherwise adapted to be associated with, a microscopic element, such as an optical microscope. In certain embodiments, imaging includes forming a two- or three-dimensional image of a portion of a sample (e.g., a spatial image of the sample). In certain embodiments, imaging includes gathering spectral data with or without the forming of a two- or three-dimensional image of a portion of the sample. In certain embodiments, imaging includes merely observing a portion of the sample, with or without forming an image of the sample.

Accordingly, in certain embodiments, the chamber of the apparatus (or the sample containing element) may be positioned on the stage of a microscope, such that the sample inside the chamber or sample containing element is bombarded by photons or electrons or other radiation, and the resulting photons, electrons or other particles of radiation, after contacting the sample or passing through the sample chamber, pass out of the chamber and are collected and analyzed to form images of the sample or other data sets which record and/or describe one or more aspects of the structure and composition of the sample. Information so collected may contain 2-dimensional or 3-dimensional spatial information, spectral (wavelength or frequency) information, compositional (e.g. chemical) information, or any other information which describes the state of the sample.

In certain embodiments, the information describing the sample results from scattering of photons or other particles, or from absorption of photons or other particles, or from changing the polarization state of photons or other particles. In certain embodiments, the information describing the sample results from emission of photons or other particles from within the sample (e.g. from fluorescence or from stimulated emission). In certain embodiments, the information describing the sample results from emission caused by multiple photon absorption (e.g. two-photon microscopy).

For instance, where a transparent material (e.g., diamond) is used to fabricate one or more of the opposing surfaces of the chamber (e.g., of a diamond anvil cell), the entire chamber (which may be inclusive of the opposing surfaces) may be mounted on the stage of a light microscope and the sample within the chamber (e.g., within the sample containing element) may be observed and/or imaged. Accordingly, the facets of the chamber (e.g., the diamond surfaces) may act as optical windows through which the sample may be observed and/or imaged.

Alternatively, the sample and/or sample containing element may be taken out of the chamber and placed directly on a light microscope stage for observation and/or imaging and/or manipulation. During all these activities, the sample may be kept frozen at low temperatures, e.g., temperatures low enough to ensure no ice crystal formation takes place within the sample (e.g., at or below the glass transition temperature of the sample) and while maintained in a manner so as to a allow a large number of photons to contact the sample. Additionally, the apparatus may be configured to allow observing and/or imaging and/or manipulating the sample while in the thawed state within or outside the chamber.

An apparatus of the invention may have any configuration so long as it includes a chamber for holding a sample that can withstand a high pressure and includes both a means of generating a high pressure within the chamber and a means for rapidly transferring heat to and from the chamber (i.e., for thawing or freezing a sample). Accordingly, an apparatus of the invention can be fabricated from a wide variety of materials, as is known in the art, but should be fabricated out of materials that can withstand both high pressure and rapid changes in extreme temperatures. The general construction and operation of anvil-type high pressure chambers are well known in the art and disclosed in the publications which are expressly incorporated in their entirety herein by reference below.

The following references discuss the design, construction and use of high pressure chambers: Ruoff et al, “The Closing Diamond Anvil Optical Window in Multimegabar Research”, J. Appl. Phys., 69 (9), 6413-6415, May 1, 1991; Mao et al, “Optical Transitions in Diamond at Ultrahigh Pressures”, Nature, vol. 351, 721 et seq, Jun. 27, 1991; Phil M. Oger, Isabelle Daniel, Aude Picard, Development of a low-pressure diamond anvil cell and analytical tools to monitor microbial activities in situ under controlled P and T, Biochimica et Biophysica Acta v1764 p 434-442 (2006); Isaac F. Silvera and Rinke J. Wijngaarden, Diamond anvil cell and cryostat for low-temperature optical studies, Review of Scientific Instruments v56 n1 p 121-124 (January 1985). R. Letoullec, J. P. Pinceaux and P. Loubeyre, The Membrane Diamond Anvil Cell: A New Device for Generating Continuous Pressure and Temperature Variations, High Pressure Research v1 p 77-90 (1988); H. Tracy Hall, High Pressure Methods, in High Temperature Technology, p 145-156, 335 & 336, McGraw-Hill, New York (1960); High Pressure Microscopic Cell PC400-MS, Teramecs Co., Ltd. Special Device Division, Kyoto, Japan (2006); Elena Müller, Detailed Investigations into the Propagation and Termination Kinetics of Bulk Homo- and Copolymerization of (Meth)Acrylates, doctoral dissertation, Mathematics and Science Faculty, Gottingen University (2005); Marcus Nowak, Harald Behrens and Wilhelm Johannes, A new type of high-temperature, high pressure cell for spectroscopic studies of hydrous silicate melts, American Mineralogist v81 p 1507-1512, (1996); K. Pressl, M. Kriechbaum, M. Steinhart and P. Laggner, High pressure cell for small- and wide-angle x-ray scattering, Review of Scientific Instruments v68 n12 p 4588-4592 (December 1997); M. Steinhart, M. Kriechbaum, K. Pressl, H. Amenitsch, P. Laggner and S. Bernstorff, High-pressure instrument for small- and wide-angle x-ray scattering. II. Time-resolved experiments, Review of Scientific Instruments v70 n2 p 1540-1545 (February 1999); N. Dahan, B. Barrau, G. Pinzutti, J. Moszkowski and G. Martinez, High-pressure design for optical measurements, Journal of Physics E: Scientific Instruments v15 n5 p 587-590 (May 1982); Joachim D. Müller and Enrico Gratton, High-Pressure Fluorescence Correlation Spectroscopy, Biophysical Journal v85 p 2711-2719 (2003). All of which are incorporated by reference in their entirety.

To better understand an apparatus of the invention, a specific embodiment of a high pressure chamber in operative communication with two opposing anvils, a gasket sample holder, a temperature modulator, and an imaging element is set forth herein below. Although the following description is set forth with reference to a particular embodiment of an apparatus of the invention for use in accordance with the methods of the invention, it is to be understood that an apparatus of the invention and its components can have a variety of configurations as will be understood by those of skill in the art.

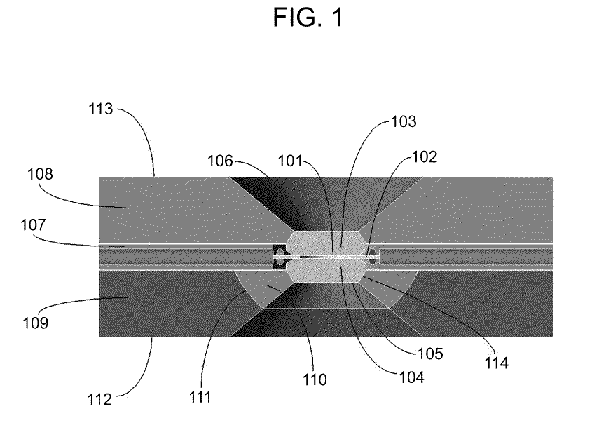

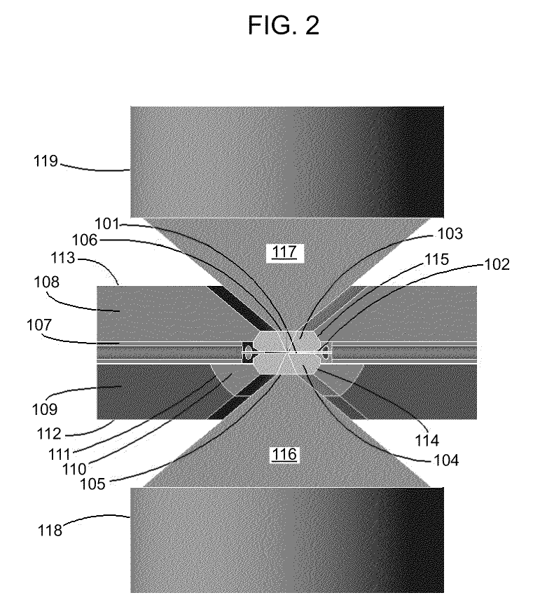

As can be seen with reference to FIGS. 1 and 2, in certain embodiments, an apparatus of the invention (100) contains a pressure modulator that includes both a force generating mechanism (e.g., a compression mechanism, not shown) and two opposing elements configured as anvils (103 and 104) (e.g., diamond disks). In this embodiment, each of the diamond anvils has an overall diameter of approximately 6 mm and a thickness of approximately 1.8 mm. In certain embodiments, the diamonds of the diamond anvils are of sufficient clarity and cut that they function as windows, capable of engaging a sample holding element, as well as allowing the transmission of photons through the various facets of the diamond so as to allow visualization of a sample contained within the sample holding element. In this embodiment, photons in the bottom cone of light (116) emerge from the photon source (118), enter the sample chamber (101) by passing through facet (105), contact or pass through the sample contained in the chamber (101), leave the sample chamber and pass through facet (106), and enter the objective (119) of the observing system via the upper cone of light (117).

The apparatus (100) further includes a sample holding element (102) (e.g., a copper gasket). In this embodiment, the sample holding element (102) is a gasket in the shape of a washer approximately 200 μm thick with an internal diameter of approximately 4 mm and an external diameter of approximately 8 mm. The two diamond anvils (103 and 104) engage the sample holding element in a manner sufficient to enclose a sample in the center of the gasket (102) thereby forming a sample chamber (101).

The pressure modulator may further include one or more pressure plates. For instance, in certain embodiments the compression mechanism of the pressure modulator may be configured to apply uniaxial perpendicular compressive forces to the outer surfaces (112 and 113) of two circular metal alloy (e.g. tungsten carbide) pressure plates (108 and 109) such that the applied forces are transmitted to the anvils (103 and 104) via mating surfaces (114 and 115). The applied forces push the anvils together, thereby compressing the sample and the gasket (102) surrounding the sample, and thereby modulating the pressure inside the sample chamber (101). The pressure plates hold the anvils in position relative to the gasket (102), with the anvil bases contacting the sample parallel and facing one another.

The bottom pressure plate (109) may contain a spherical bearing (110) with a spherical bearing surface (111) that allows the bottom diamond anvil (104) to automatically position itself parallel to the top diamond anvil (103) as forces are applied to surfaces (112 and 113) of the pressure plates. A thin layer of soft metal (e.g. lead foil) may be inserted at the mating surfaces (114 and 115) between the pressure plates (108 and 109) and the anvils (103 and 104), to ensure that the forces applied to the anvils by the pressure plates are applied evenly.

The apparatus (100) additionally includes a temperature modulator configured for modulating the temperature of the chamber from a temperature that is below the freezing point of water to a temperature that is above the freezing point of water, or alternatively from a temperature that is above the freezing point of water to a temperature that is below the freezing point of water.

The temperature modulator includes a heating source, a cooling source, one or more delivery conduits and one or more delivery mechanisms. The heating source may be a fluid reservoir for containing and heating a fluid, such as a gas (e.g., helium) or liquid (e.g., water). The heating source may further be connected to an electrical source. The cooling source may be a fluid reservoir for containing and cooling a fluid, such as a cryogenic fluid (e.g., liquid nitrogen).

The delivery conduit is configured for delivering a heated or cooled fluid to the delivery manifold (107). The delivery conduit may be connected to only the heating source, to only the cooling source, or to both. Accordingly, the delivery conduit may be one or a plurality of tubes, pipes, or the like, connected to the delivery manifold. The delivery conduit may be fabricated from any material capable of transporting fluids and withstanding extreme temperatures. For instance, the delivery conduit may be fabricated from plastic, glass, metal or the like.

The delivery mechanism may be a manifold (107) that is configured for receiving the heated or cooled fluid from the one or more delivery conduits and delivering the received fluid to the apparatus of the invention in a manner sufficient to heat or cool the other components of the device, for instance, the anvil(s) (103 and/or 104) and/or the sample holding element (102). The delivery manifold may be configured to be prewarmed or precooled by passing warm or cold fluid through passages in the manifold which do not deliver fluid into contact with the anvils (103 and 104) or the sample holding element (102) Specifically, the delivery manifold (107), as shown, may be configured for contacting one or more of the pressure modulators (e.g., one or more anvils 103 and 104 thereof) and the sample holding element (102) with a heating or cooling fluid of the invention and thereby heating or cooling the sample chamber (101) and its contents (e.g. a biological sample).

Accordingly, within the sample chamber (101) an enclosed sample (e.g. a biological sample) can be thawed and/or frozen in accordance with the methods of the invention (e.g. rapidly under controlled pressure and temperature conditions) and imaged within the sample chamber (101) or imaged once removed from the sample chamber.

Although with respect to the illustrated embodiment, the temperature modulator is configured for heating or cooling a sample by contacting a chamber containing the sample and thereby heating or warming the sample, it is to be noted, that other configurations for producing a high pressure chamber and/or heating and/or cooling the sample may also be provided as is well known in the art and described above.

For instance, in one embodiment, the anvil includes at least one gem stone, for instance, a diamond and a post, for instance, a metal post. In certain embodiments, the gem stone anvil (e.g., diamond) and the post interact with a sample containing element to produce a sample or pressure chamber. For example, in one embodiment, a sample chamber may include a disk (e.g., a metal disk, such as copper) for containing a sample. The disk may contain a depression in which the sample is placed. This disk may be placed between a single diamond anvil and a metal post to produce a pressure chamber. The diamond anvil may be configured to contact the sample and cover the depression in the disk thereby enclosing the sample in the depression in the disk. The sample may thereby be sealed inside the depression in a pressure tight seal.

In accordance with this embodiment, the post may contain a hole. The post may further contain a fluid. For instance, a liquid or a gas may be contained within the hole of the post. The bottom surface of the disk may be positioned to cover the hole in the post and pressure may be applied to the sample by means of the fluid in the hole in the post. For example, when pressure is applied to the fluid, the pressure is transmitted to the bottom of the metal disk containing the sample. The pressure may then deform the bottom of the metal disk and pressurize the sample in the sample chamber. After pressurizing the sample chamber, the temperature modulating fluid may be applied to the outside of one or more of the diamond anvil, the metal disk and/or the metal post.

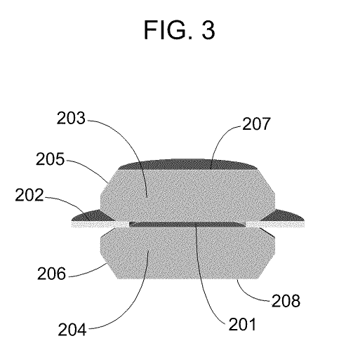

FIG. 3 is a cutaway diagram showing a representative sample cell of FIG. 1. The sample chamber (201) is enclosed between the diamond windows (203 and 204), and surrounded on the edge by gasket (202). Mating surfaces (205 and 206) transmit force to the diamond windows, and light may be passed through diamond window surfaces (207 and 208) to allow illumination and observation of the sample chamber.

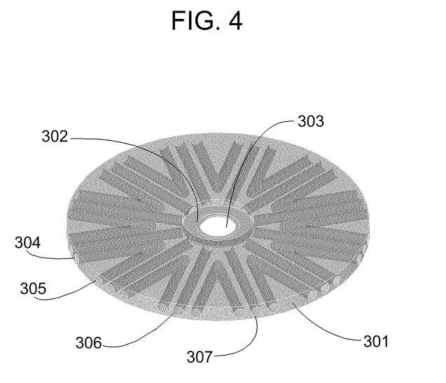

FIG. 4 is a diagram showing a representative delivery manifold (107 of FIG. 1) located between the pressure plates and surrounding the sample cell. Referring to FIG. 1, the manifold (107) may be of any shape or size, for instance, square, hexagonal, circular or the like, but the thickness of the manifold is such that, when compressive force is applied to surfaces (112 and 113) to compress the anvils (103 and 104), the pressure plates (108 and 109) do not interfere with or contact the manifold (107). In the present embodiment the manifold (301) is circular. The fluid manifold (107) may be made of any suitable material (e.g. metal or glass) through which temperature modulating fluids may be passed, to modulate the temperature of the sample cell. The manifold may contain a number of passages (e.g., 1, 2, 3, 4, 5, 10, 15, 20 or more). In this embodiment, the manifold contains six tubular passages (306) for the application of a temperature modulating fluid, and six tubular passages (307) for removal of a temperature modulating fluid from the central space containing the sample cell. In this embodiment, fluid delivery manifold also contains six each tubular passages (304 and 305) for passing temperature modulating fluids through the manifold without contacting these fluids to the sample cell, e.g., for the purposes of precooling or prewarming of the fluid manifold itself. The fluid manifold may also be operatively connected to a reservoir for containing the temperature modulating fluid. Temperature sensing devices (e.g. thermocouples, not shown) may be mounted at appropriate places in the manifold, to monitor the temperature near the sample cell. In the center of the fluid manifold is the gasket (302) and the sample chamber volume (303).

Additionally, the assembly of FIG. 1 may be mounted, or be configured to be mounted, on the stage of an optical system (e.g. a microscope) such that the optical system may illuminate and/or observe and/or image the sample by passing light or other radiation through one or more of the diamond windows. Portions of the optical system (e.g. a microscope) may also be used to illuminate the sample with laser beams or other radiation sources, to allow optical or other manipulation of the sample while the sample is inside the sample chamber.

In operation, a sample (e.g. a biological sample) is placed in the sample chamber and the upper diamond window, mounted in its pressure plate, is placed over the sample so as to seal the sample inside the sample chamber. Pressure is then applied to the sample by applying force to the pressure plates, compressing the sample and the gasket between the diamond windows. Before and/or while pressure is being applied, the fluid delivery manifold may be precooled or prewarmed by passing a cryogenic fluid (e.g. liquid nitrogen) or a warming fluid (e.g., heated water or helium gas) through the manifold precooling/prewarming passages. Once the pressure reaches a desired value, and the manifold has been precooled or prewarmed, a cryogenic fluid or a warming fluid may be applied to the sample cell by passing such fluid through the application and removal passages of the manifold. Contact of the cryogenic or warming fluid to the sample cell rapidly cools or warms the cell and freezes (e.g. vitrifies) or thaws the sample contained within it.

In the frozen (e.g. vitrified) or thawed state, the sample may be observed and/or imaged and/or manipulated, while in the chamber, using an optical system (e.g. a light or optical microscope) or by other optical or microscopic means (e.g., via infra-red or x-ray or electron microscopy), for arbitrarily long periods of time, for instance, to a allow a large number of photons, electrons, or the like, to contact the sample.

Contacting a large number of photons with the sample over a prolonged period of time reduces photon noise without requiring high overall light (e.g., photon) intensity or irradiance, which is an important factor when visualizing (e.g., imaging) a sample (e.g. a biological sample) in a manner so as to maintain the structural integrity and/or viability of the sample. If the intensity or irradiance of the light is too great, structural and cellular perturbation increases and the structural integrity and/or viability of the sample may be compromised.

Thus, in certain embodiments, the methods of the invention allow for the detailed ultra-structural observation and imaging of a viable sample (e.g., via optical microscopic means, such as structured light microscopy) so as to generate a super resolution (e.g. better than Rayleigh criterion) image of the sample while preserving the viability and/or structural integrity of the sample. In certain embodiments, this may be achieved by increasing the time period over which the light (e.g., photons) is contacted with the sample and thereby increasing the number of photons which contact the sample and thereby increasing the maximum attainable overall signal-to-noise ratio and/or spatial resolution and/or spectral resolution of the data, without increasing the photon intensity or irradiance.

In certain other embodiments, an apparatus of the invention is characterized in that it is configured for both freezing and/or thawing a biological sample inside a chamber of a high pressure modulator in a manner sufficient to maintain the structural integrity and/or viability of the biological sample, and for removing the sample from the high pressure modulator for imaging and/or manipulating the sample, while the sample is outside of the high pressure modulator, and then returning the sample to the high pressure modulator for refreezing or rethawing after observing and/or imaging and/or manipulating the sample.

For instance, an apparatus of the invention may include a transfer element, such as a robotic arm which can transfer the sample while frozen or thawed and still contained within the chamber or sample containing element (e.g., an annular enclosing gasket) from the location of the high pressure modulator or chamber, to the viewing stage of an optical microscope. In this embodiment, the microscope viewing stage may be kept at cryogenic or warming temperatures so that the sample remains frozen or thawed (e.g., at ambient temperatures) while it is observed by means of the microscope, and the portion of the robot arm which contacts the sample may also be kept at cryogenic or warmed (e.g. ambient) temperatures so that the sample remains frozen or thawed during the transfer. In certain embodiments, the sample may be returned to the chamber after viewing and/or manipulation (if desired) by the same or a different transfer element (e.g., a different robotic arm mechanism).

In certain embodiments, the sample and the opposing surfaces of the pressure modulator (e.g., the diamond anvils) may be separated with the aid of chemical parting substances (e.g. lecithin or 1-hexadecene) coating the surfaces of the diamond anvils which contact the sample, as is well known in the art. In certain embodiments, the sample chamber may be opened by lowering the bottom pressure plate and enclosing gasket, which causes the bottom anvil and the enclosing gasket, containing the sample, to part contact with the upper anvil. When the bottom portion of the apparatus containing the bottom anvil and the sample and gasket have cleared the top portion, a robotic arm may be engaged to contact and grasp the gasket and the sample contained within it and then move the sample onto the cryogenic microscope stage for viewing or manipulation. In certain embodiments, the bottom anvil is also carried to the cryogenic microscope stage along with the gasket and sample. In certain embodiments, the sample and gasket and lower anvil remain stationary, and the microscope objective is moved into place over the sample, after the top portion of the apparatus is removed.

In another embodiment, the apparatus is configured so that the sample may be removed from the enclosing gasket for viewing. In this configuration, the inner surface of the gasket may be shaped in the form of a truncated cone, so that the enclosed sample may be more easily removed from the gasket by lifting the sample in the direction of the big end of the cone, while the gasket is lifted in the opposite direction. In this embodiment, the inner surface of the gasket may be coated with chemical parting substances as mentioned above.

In embodiments in which it is desired to return the sample to the pressure chamber after observation or modification, the chamber may be filled with a cryogenic liquid (e.g. liquid nitrogen) or a warming fluid before it is closed, to fill up any spaces in the chamber not occupied by the sample, so that hydrostatic pressure may be reestablished after the chamber is closed. In certain embodiments, filling the chamber this way may be accomplished by closing the chamber under cryogenic liquid or warming fluid.

After observation, imaging and/or manipulation are completed, the sample, in the sample cell, may be rewarmed or re-frozen by applying pressure (if not already pressurized) to the chamber and, while pressurized, applying rewarming or cooling fluids, in appropriate sequence and timing, to the sample cell. These rewarming or cooling fluids may be applied to the sample cell by passing them through the application and removal passages in the prewarmed/precooled fluid manifolds.

Accordingly, the methods of the invention allow for the acquisition of detailed, high resolution images (e.g., optical microscopic images) of the collected sample (e.g., a cellular, tissue or multicellular organism sample). Because the sample is frozen (e.g., vitrified or cryogenically fixed) and/or thawed with the production of few or no ice crystal artifacts, ultra-structural details of the sample and/or the cells within the sample and/or the components within the cells may be observed, imaged and otherwise analyzed with little or no interference due to such artifacts. The observation, imaging and/or analysis may be performed while the sample remains frozen (e.g., while the chemical, biochemical and molecular processes of the cell are ceased) or while the sample is thawed.

Accordingly, in the cryogenically fixed state, the observation, imaging and/or analysis of the sample may be performed via optical microscopy over a prolonged period of time in a manner sufficient to allow a large number of photons to be contacted with and/or passed through the sample and thereby to produce one or more high signal-to-noise ratio, high resolution images or data sets of the sample. In the cryogenically fixed state, photons may be made to contact or pass through the sample over arbitrarily long periods of time, ranging from one minute to three minutes, or from 10 seconds to 30 minutes, or from 1 second to 24 hours, or from 100 milliseconds to 30 days, or from 10 milliseconds to 1 year, or for any arbitrarily long period of time. For instance, the image acquisition process herein described may be used to obtain detailed structural information about the sample, the cells of the sample or the various components within the cells, such as the location, orientation and composition of sub-cellular structures of the cells of the sample as well as the cell to cell structure of the overall tissue.

For example, in one embodiment, the tissue to be imaged and/or manipulated is from an organ (e.g., a brain) and the tissue of interest (e.g., neural tissue) is excised from that organ in a manner sufficient to preserve the viability of the sample, as is well known in the art. Accordingly, the organ (e.g., a brain) from which the tissue (e.g., neural tissue) is to be harvested may first be put into a state of cold but not frozen suspended animation (e.g., at a temperature between 273 K and 283 K) and then carefully sliced in a manner to reduce damage to the tissue sections collected, as is well known in the art. The sliced sections may be from about 10 μm to about 300,000 μm, such as from about 20 μm to about 1000 μm, e.g., from about 200 μm to about 400 μm. The tissue (e.g., neuronal cells) collected may then be placed into a chamber of a device of the invention.

The sample is placed within the chamber and the temperature and pressure within the chamber are modulated to cause the freezing of the sample with minimal to no ice crystal formation within the sample (e.g., both within and between the cells of the sample). The freezing of the sample may take place rapidly, and in a manner such that the sub-cellular structures and their positioning remains unaffected (e.g., by ice crystal formation) and the cell to cell alignment within the tissue remains intact. In certain embodiments, the freezing of a biological sample takes place in a manner such that the chemical, biochemical and molecular processes within the biological sample cease.

After the sample has been frozen in a manner sufficient to fix (e.g. immobilize) the sample without compromising the structural integrity of the majority of the components of the sample and/or without compromising its viability, the sample may then be manipulated and/or imaged (e.g., analyzed) in any of a number of ways over a short or long period of time, for instance, while the sample remains frozen during the manipulating and/or imaging and/or analyzing. The sample may be manipulated or perturbed in a number of ways while in the frozen state, including: physical, chemical, electrical, optical, molecular, and nanotechnological perturbation. While in the frozen state, cells may be added to or removed from the sample, or subcellular components may be added to or removed from the cells of the sample. The sample may be manipulated and/or imaged either while still in the sample chamber or after having been removed from the sample chamber, as described above.

Where multiple samples are collected from a single organ or organism, multiple images of each of the samples may be collected (e.g., via a suitable detector), stored and analyzed, for instance, via computer means. Such multiple images may then be combined to gain detailed knowledge of extended portions of the organ or organism from which the samples were collected. For instance, a complete detailed image (e.g., a three dimensional digital image) of an organ (e.g., a brain) and its structure(s) may be obtained, stored, examined, reproduced and otherwise analyzed to give detailed information of the structure of the organ, how the organ works, and how the individual cells (e.g., neurons) interact or associate with one another within the organ.

Although, the above has been described with respect to determining the structure and/or function of an organ this should not be construed as limiting the scope of the invention in any way as modifications to the above description may be made without diverging from the invention. For instance, the above methods may be used to characterize the contents and interactions between the various components of a cell or other sub-cellular structure (e.g., nucleus, chromosomes, etc.), or between the components of a portion of an organ (e.g. a brain or other neural tissue).

A frozen manipulated and/or imaged and/or analyzed the sample may be thawed in a manner sufficient to maintain the structural integrity and/or viability of the sample. Accordingly, to thaw a frozen sample, the sample is placed within a chamber of the apparatus (if not already therein). Once in the chamber, the temperature and pressure inside the chamber may be modulated in a manner sufficient to cause the thawing of the sample with minimal to no ice crystal formation within the sample (e.g., both within and between the cells of the sample). The thawing of the sample may take place rapidly, as described above, and in a manner such that the sub-cellular structures and their positioning remains relatively unaffected (e.g., due to the melting and/or recrystallization of fluid components within and between the cells of the sample) and the cell-to-cell alignment within the tissue remains intact. Once thawed the cells of the sample maintain their viability and structural integrity and continue their typical cellular processes.

After a sample has been thawed in a manner sufficient to maintain the structural integrity of the majority of the components of the sample and/or to maintain its viability, the sample may then be analyzed via optical microscopy, or otherwise imaged and/or manipulated, either while still in the sample chamber or after having been removed from the sample chamber. For instance, where the sample is a viable biological sample, once thawed, the viable sample or one or more of the viable cells of the sample may be manipulated or perturbed in a number of ways well known in the art, including: physical, chemical, electrical, optical, molecular, and nanotechnological perturbation. Individual or multiple cells may be added or removed from the sample. Subcellular components of cells may be modified, or added to or removed from the cells of the sample.

After manipulation the sample may then be refrozen in the manner described above, i.e., in a manner sufficient to maintain the structural integrity and/or the viability of the sample, and observed and/or imaged and/or manipulated while in the frozen state. In this way, one or more biological (e.g., cellular) processes may be observed and imaged in a living or viable cell or tissue over time and over one or more (e.g., several) cycles of freezing and thawing, wherein the cell is observed and/or manipulated in some manner, frozen, observed and/or manipulated, thawed, again observed and/or manipulated in some manner, re-frozen, observed and/or manipulated, etc.

Accordingly, because when the sample is in the frozen state, the cellular structures are immobilized and cellular processes of the viable cells of the sample are arrested, the effects of a previously applied perturbation can be observed and imaged in great detail and with high spatial and/or spectral resolution, because such observation or imaging may be accomplished over arbitrarily long periods of time, allowing high signal-to-noise ratios to be attained in the data sets, as described above. In one embodiment, the manipulated sample is frozen to produce a frozen viable sample following the manipulation, which may then be observed and then re-thawed in a manner sufficient to maintain the structural integrity and/or viability of the sample.

Hence, using the methods disclosed herein, one is capable of observing a tissue or a cell, manipulating the tissue or cell, freezing the tissue or cell so as to cryogenically fix (e.g., arrest) the tissue or cell image and observe the cellular changes that have taken place after the manipulation and before the cryofixation (e.g., via light microscopy, as described above) and then thaw the tissue and cell while maintaining is structural integrity and vitality. This process may be performed once or performed repeatedly over several cycles of freezing and thawing. Using the methods disclosed herein, one is capable of manipulating a tissue or cell in the frozen state, and observing the changes that have taken place after the manipulation while still in the frozen state, and after subsequent thawing.

The methods described herein may be used to observe the time evolution of one or more cells or cellular processes. Accordingly, individual cellular structures and/or identifiable chemical components (e.g., components labeled with an observable dye that does not compromise the viability of the cell) can be observed using super-resolution imaging techniques (e.g. structured light microscopy) and followed over time and over several cycles of manipulation, freezing, manipulation and/or observation, and thawing. It is to be noted that although the observation methods disclosed herein have been described with respect to observing a frozen sample, the thawed sample may also be observed as part of the experimental process. Repeated high resolution imaging of the same cellular structures, combined with the ability to perturb those structures as desired, may allow greater understanding of the relationship of structure to function in biological samples.

For instance, the methods herein described are useful in manipulating and imaging living cells contained on silicon chips. Mammalian cells, such as nervous system cells (e.g., neurons) may be associated with a silicon microchip so as to form a combination biological and electronic circuit, as is well known in the art. For instance, in certain embodiments, one or more cells (e.g., a cell, a neuron, a gamete cell, stem cell, or the like) may be associated with a substrate, for instance, a glass, silicon or electronic chip so as to form a biological circuit. For example, the one or more cells may be arranged in a circuit configuration and may interact with other circuitry components to form a circuit that is capable of transferring a current or other signal from one point on the chip to another. In one embodiment, the microchip substrate is configured for being contained within a chamber of the high pressure apparatus and is capable of being moved into and out of the chamber and/or for being associated with a stage of an imaging device for imaging the components (e.g., the biological components) of the microchip (e.g., the associated biological cells).

The association of a biological cell with a substrate so as to form a microchip that contains biological components is well known in the art and disclosed in following references, which are hereby incorporated by reference in their entirety for their teaching on the production and use of biochips. The Neurally Controlled Animat: Biological Brains Acting with Simulated Bodies. Thomas B. DeMarse, Daniel A. Wagenaar, Axel W. Blau and Steve M. Potter Autonomous Robots v.11 n.3 p. 305-310 (November 2001). Noninvasive neuroelectronic interfacing with synaptically connected snail neurons immobilized on a semiconductor chip Günther Zeck and Peter Fromherz PNAS|Aug. 28, 2001, vol. 98 no. 18 p. 10457-10462. Engineering a biospecific communication pathway between cells and electrodes Joel H. Collier and Milan Mrksich PNAS|Feb. 14, 2006, vol. 103 no. 7 p. 2021-2025. Closing the Loop: Stimulation Feedback Systems for Embodied MEA Cultures S. M. Potter, D. A. Wagenaarand T. B. DeMarse. In: Advances in Network Electrophysiology Using Multi-Electrode Arrays, M. Taketani and M. Baudry (Eds.), Springer (2005). For instance, cortical neurons from a suitable organism may be dissociated and cultured on a surface containing a grid of electrodes (multi-electrode arrays, or MEAs) capable of both recording and stimulating neural activity.

Such microchips containing biological circuits may be useful in studying the behavior of neurons, in analyzing the information processing functions of particular neurons or samples of neural tissue, as detectors for environmental pathogens or toxins, as drug screening systems, as chemical sensors (artificial noses), for development of medical devices such as neural prostheses, for the generation of organic computers using living neurons, and for other applications. Accordingly, the methods of the invention are useful for imaging, analyzing and/or manipulating the neuron containing biochips once they have been fabricated, or during their fabrication as part of the fabrication process. Hence, the methods herein disclosed are useful in both studying the effects of and implementing modifications to biochips containing neurons or other cells.

In another aspect, the present invention is directed to a computer program that may be utilized to carry out the above steps. The device of the invention may include mechanisms to open and close the sample chamber, place the sample into and remove the sample from the chamber, control the application of forces applied to the pressure plates, monitor and control the application of the cooling and warming fluids, and operate various devices (e.g., the robotic arm and/or imaging apparatus) to manipulate and/or observe the sample either inside or outside the chamber. One or more of the steps taken to operate these mechanisms, including: the placement of a sample into the chamber, the alignment of the opposing surfaces of the pressure plates, the enclosing of the chamber, the generation of a force, the modulation of the pressure within the chamber, the modulation of the temperature of the chamber, the placement of the sample and/or chamber components onto an observation stage, the observing (e.g., imaging) and/or manipulation of the sample, in accordance with the invention, may all be done automatically under computer control, that is, with the aid of a computer. The computer may be driven by software specific to the methods described herein. Examples of software or computer programs used in assisting and conducting the present methods may be written in any convenient language, e.g. Visual BASIC, FORTRAN and C++ (PASCAL, PERL or assembly language), and may run in the environment of any suitable operating system, e.g. LINUX, UNIX, Mac OS or Windows. It should be understood that the above computer information and the software used herein are by way of example and not limitation.

Programming according to the present invention, i.e., programming that allows one to carry out the methods of the invention, as described above, can be recorded on computer readable media, e.g., any medium that can be read and accessed directly by a computer. Such media include, but are not limited to: magnetic storage media such as floppy discs, hard disc storage medium, and magnetic tape; optical storage media such as CD-ROM and DVD; electrical storage media such as RAM and ROM; and hybrids of these categories such as magnetic/optical storage media.

In certain embodiments, a processor of the subject invention may be in operable linkage, i.e., part of or networked to, the aforementioned apparatus, and capable of directing its activities. A processor may be pre-programmed, e.g., provided to a user already programmed for performing certain functions, or may be programmed by a user.

Thus, in certain embodiments, the programming is further characterized in that it provides a user interface, where the user interface presents to a user the option of selecting among one or more different, including multiple different, rules for individually controlling the steps of the methods herein disclosed. A processor may be remotely programmed by “communicating” programming information to the processor, i.e., transmitting the data representing that information as electrical signals over a suitable communication channel (for example, a private or public network). Any convenient telecommunications means may be employed for transmitting the programming, e.g., facsimile, modem, Internet, LAN, WAN or other network means, wireless communication, etc.

It is evident from the above discussion that the subject invention provides an important breakthrough in the manipulation and imaging and observation of biological samples with high resolution and with high levels of detail and control, and with reduced levels of undesired degradation of the structural integrity and/or viability of the sample. Specifically, the subject invention allows one to image, with very high spatial and/or spectral resolution, the internal structures and processes of a cell, as well as the external milieu of a tissue sample, sequentially over a prolonged period of time, without unduly compromising the viability of the cell. Accordingly, the subject invention represents a significant contribution to the art.

All publications and patents cited in this specification are herein incorporated by reference, in their entirety, as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method, comprising:
thawing a high pressure frozen biological sample under pressure in a manner sufficient to maintain structural integrity of said biological sample to produce a thawed sample; and
imaging said thawed sample.

2. The method of claim 1, further comprising manipulating said thawed sample.

3. The method of claim 2, wherein said manipulation is selected from the group consisting of a physical, chemical, optical, molecular, and a nanotechnological manipulation.

4. The method of claim 2, further comprising imaging said thawed sample after said manipulation.

5. The method of claim 2, further comprising freezing said thawed sample under pressure after said manipulating to produce a frozen sample.

6. The method of claim 5, further comprising imaging said frozen sample.

7. The method of claim 5, wherein said freezing of said thawed sample is performed in a manner so as to maintain the viability of the sample to produce a frozen viable sample following said manipulating.

8. The method of claim 7, further comprising, thawing said frozen viable sample under pressure in a manner sufficient to maintain the structural integrity and/or viability of said sample.

9. The method of claim 6, further comprising manipulating said frozen viable sample.

10. The method of claim 9, wherein said manipulation comprises removing an element of the sample or adding one or more elements to the sample.

11. The method of claim 8, wherein said manipulation is selected from the group consisting of a physical, chemical, optical, molecular, and a nanotechnological manipulation.

12. The method of claim 1, wherein said thawing is performed in a manner sufficient to maintain the viability of said sample.

13. The method of claim 1, wherein said imaging is performed by an optical microscope.

14. The method of claim 1, wherein said biological sample comprises multiple cells.

15. The method according to claim 12, wherein said biological sample comprises a tissue sample.

16. The method according to claim 1, wherein said biological sample comprises a multicellular organism.

17. The method according to claim 1, wherein said biological sample comprises one or more ova or spermatozoa.

18. The method according to claim 1, wherein said biological sample comprises one or more embryos.

19. (canceled)

20. The method according to claim 1, wherein said biological sample comprises one or more cells associated with a substrate.

21. An apparatus comprising:
a chamber having an interior configured to hold a sample;
a pressure modulator for modulating the pressure of said interior;
a temperature modulator for modulating the temperature of said interior from a temperature that is below the freezing point of water to a temperature that is above the freezing point of water; and
an imaging element for imaging said sample.

22-37. (canceled)
